(19) United States
(12) Patent Application Publication
(10) Pub. No.: US 2011/0295198 A1
(43) Pub. Date: Dec. 1, 2011

(54) SYSTEME DE PREPARATION ET/OU D'ADMINISTRATION DE FLUIDES DE TRAITEMENT MEDICAL

(75) Inventor: Philippe BUISSON, TOUSSIEU (FR)

(73) Assignee: DORAN INTERNATIONAL, TOUSSIEU (FR)

(21) Appl. No.: 13/114,249

(22) Filed: May 24, 2011

Related U.S. Application Data
(60) Provisional application No. 61/347,966, filed on May 25, 2010.

(51) Int. Cl. A61M 5/00

(52) U.S. Cl. 604/83; 604/256

(57) ABSTRACT

The system comprises a transport module (3) in which are housed administration reservoirs (6) each intended to contain a medical treatment fluid, an administration line (7) equipped with a fluid inlet and outlet (8, 9) accessible from the outside of the transport module and to which is connected each administration reservoir, and obturation means (12) associated with each administration reservoir, the obturation means (12) associated with each administration reservoir being moveable between first and second positions in which the corresponding administration reservoir is fluidically isolated and fluidically connected to the administration line respectively, and a preparation and/or administration unit on which the intention is to position the transport module (3), the preparation and/or administration unit including first control means laid out for controlling the displacements of the obturation means (12) associated with each administration reservoir (6) between their first and second positions.

The present invention relates to a system for preparing and/or administering medical treatment fluids.

A perfusion line for administering medical treatment fluids to a patient, such as chemotherapy products, include in a known way:
a valve comprising a fluid inflow route including means for connecting to a syringe, and a fluid outlet route, and
an extension, one of the ends of which, a so-called proximal end, is mounted on the fluid outlet route and the other end of which, a so-called distal end, is equipped with means for connection to a catheter.

The method for using a such perfusion line comprises the following steps:
connecting a first rinsing syringe containing a rinsing product, such as normal saline or glucose to the fluid inflow route of the valve,
purging the valve and the extension by injecting into the latter a sufficient amount of rinsing product contained in the rinsing syringe,
disconnecting the rinsing syringe,
connecting an administration syringe containing a medical treatment fluid to the fluid inflow route of the valve,
injecting the medical treatment fluid as far as the catheter with the administration syringe, and
disconnecting the extension of the catheter.

The use of such a perfusion line involves steps which are dangerous for hospital personnel when the medical treatment fluid to be administered to the patient is a chemotherapy product, notably because of the handling of the containers containing these products by hospital personnel, and risks of projections of chemotherapy product or of inspiration of vapors of this product during the disconnection of the extension of the catheter.

Further, during the transport of a chemotherapy product between its preparation and administration areas, the container containing the latter may be broken or contaminated, and the carrier may come into contact with chemotherapy product particles deposited on the outer walls of the container.

Further, because the chemotherapy product is prepared a long time before its administration to a patient, it may be mandatory for the hospital personnel to shake the container containing this product in order to ensure a homogeneous mixture of the different constitutive products of the latter, which increases the risk of contamination of hospital personnel and the risks of faulty administration to the patient.

Document DE 43 36 336 discloses a system for administering medical treatment fluids comprising a device for actuating a pump and a valve B2 on which are mounted a cartridge equipped with an infusion pump A2, a cartridge forming a collector D, and a cartridge for applying drugs E.

The device for actuating a pump and valve B2 notably comprises a plurality of administration reservoirs 101-105 mounted on the outer face of the pump and valve actuation device, first and second control means laid out in order to control the opening and closing of the different valves respectively fitting out the cartridge for applying drugs E and the cartridge forming a collector D, and third control means laid out in order to control the compression of the compression chamber 47 and the emptying of the latter.

The administration system described in document DE 43 36 336 substantially has the same drawbacks as the perfusion line described earlier, notably because of the mounting of the administration reservoirs on the outer face of the pump and valve actuation device.

The present invention aims at finding a remedy to all or part of these drawbacks.

The technical problem at the root of the invention therefore consists of providing a system for preparing and/or administering medical treatment fluids which is of a simple and economical structure, and the use of which gives the possibility of protecting the hospital personnel and a carrier from any contact with the treatment liquids intended to be administered to a patient.

For this purpose, the invention relates to a system for preparing and/or administering medical treatment fluids comprising:
at least one transport module in which are housed at least one administration reservoir intended to contain a medical treatment fluid to be administered to a patient, an administration line fitted out with a fluid inlet and outlet accessible from the outside of the transport module and to which is connected each administration reservoir, and obturation means associated with each administration reservoir, the obturation means associated with each administration reservoir being moveable between a first position in which the corresponding administration reservoir is fluidically isolated from the administration line and a second position in which the corresponding administration reservoir is fluidically connected to the administration line, and
at least one preparation and/or administration unit on which it is intended to position the transport module, the preparation and/or administration unit including first control means laid out for controlling the displacements of the obturation means associated with each administration reservoir between their first and second positions.

The system according to the invention advantageously includes a plurality of administration reservoirs housed in the transport module.

Thus, because the different administration reservoirs are stored in the transport module, the hospital personnel and a possible carrier cannot come into contact with the medical treatment fluids to be administered to a patient, and therefore cannot be contaminated by the latter.

Further, with these steps, it is possible to protect the reservoirs during their transport between their preparation and administration areas, and also preserve the cleanliness of the latter.

It should be noted that one unit may only be used for performing the filling of the different reservoirs contained in a transport module or only for performing the administration of medical treatment fluids to a patient. In this case, the system according to the invention advantageously includes at least one unit for preparing fluids on which the intention is to position a transport module so as to fill each administration reservoir with a medical treatment fluid, and at least one fluid administration unit on which the intention is to position the transport module so as to administer to a patient the different medical treatment fluids contained in the administration reservoirs of the latter.

It should be noted that a unit may be used both for performing the filling of the different reservoirs contained in a transport module and for performing the administration of medical treatment fluids to a patient. The unit is then identified as a preparation and administration unit.

Preferably, the first control means are laid out so as to control the displacements of the obturation means associated with each administration reservoir only in an operating position of the preparation and/or administration system in which the transport module is positioned on the preparation and/or administration unit.

According to an embodiment, the first control means include monitoring means and actuation means driven by the monitoring means and laid out so as to mechanically cooperate with the obturation means associated with each administration reservoir when the transport module is positioned on the preparation and/or administration unit and for displacing the latter between their first and second positions.

Advantageously, the obturation means associated with each administration reservoir include a 3-way valve.

According to another embodiment, the first control means are laid out so as to be electrically coupled with the obturation means associated with each administration reservoir when the transport module is positioned on the preparation and/or administration unit. According to this embodiment, the transport module may include an electric contactor intended to cooperate with an electric contactor positioned on the preparation and/or administration unit when the transport module is positioned on the latter.

Advantageously the transport module is made in the form of a transport box.

Advantageously, the transport module includes a lid moveable between opening and closing positions of the transport module, and means for locking the lid which are moveable between a position for locking the lid in its closing position and a position for unlocking the lid. With these steps it is possible to prevent any handling of the administration reservoirs during their transport.

Advantageously, the preparation and/or administration unit comprises a second control means laid out for controlling the displacements of the locking means between their locking and unlocking positions.

Preferentially, the preparation and/or administration system includes means for locking the transport module on the preparation and/or administration unit.

Preferentially, the transport module further contains at least one dilution reservoir connected to the administration line upstream from the administration reservoirs and intended to contain a dilution fluid such as normal saline or glucose, and obturation means associated with each dilution reservoir, the obturation means associated with each dilution reservoir being moveable between a first position in which the corresponding dilution reservoir is fluidically isolated from the administration line and a second position in which the corresponding dilution reservoir is fluidically connected to the administration line, and in that the first control means are laid out for controlling the displacements of the obturation means associated with each dilution reservoir between their first and second positions.

Preferably, the administration line comprises a mixing chamber positioned downstream from the administration reservoirs and upstream from the fluid outlet.

According to a characteristic of the invention, the transport module includes a bypass line, one of the ends of which is connected to the mixing chamber and the other end of which is connected to the administration line upstream from the administration reservoirs and downstream from said at least one dilution reservoir, the bypass line including obturation means which are moveable between a first position in which the obturation means prevent fluid flow through the bypass line and a second position in which the obturation means allows fluid flow through the bypass line, and the first control means are laid out in order to control the displacements of the means for obturating the bypass line between their first and second positions. With these steps it is possible to perform a dilution of the fluid(s) contained in the mixing chamber before their administration to the patient, which is advantageous when the mixture is not stable over time.

Preferably, the administration line includes a volumetric pump positioned between the administration reservoirs and the mixing chamber, and a volumetric pump positioned between said at least one dilution reservoir and the administration reservoirs.

Advantageously, the transport module comprises a recovery reservoir connected to the administration line upstream from the fluid outlet, and preferably downstream from the mixing chamber, and obturation means which are moveable between a first position in which the recovery reservoir is fluidically isolated from the administration line and a second position in which the recovery reservoir is fluidically connected to the administration line, and in that the first control means are laid out so as to control the displacements of the obturation means associated with the recovery reservoir between their first and second positions.

Advantageously, the transport module is fitted out with an identification module, preferably an RFID chip, and the preparation and/or administration unit includes contactless interactive means intended to detect and identify the identification module when the transport module is positioned on the preparation and/or administration unit. With these steps it is possible to ensure traceability of the transport module.

Preferably, the preparation and/or administration unit includes a central data processing unit and data transfer means intended to transmit data characteristic of a detected and identified identification module to the central data processing unit. The transfer means may for example be wireless transfer means such as transfer means of the wifi type.

The preparation and/or administration unit advantageously includes means for time-stamping of the cycles for filling the administration reservoirs housed in the transport module, and the cycles for administering the patient medical treatment fluids contained in said reservoirs.

According to an embodiment, the preparation and/or administration unit includes a barcode reader and a barcode printer intended to print a label to be positioned on the transport module, the barcode of the latter containing data relating to the patient and to the contents of each reservoir (for example the nature and the amount of the product contained in each reservoir, the date and the time of preparation of the product). The barcode of the label to be positioned on the transport module may also contain data relating to the operator having prepared the product.

According to an embodiment, the transport module comprises pressurization means laid out in order to maintain a predertermined pressure on the outer walls of the administration reservoirs housed in the transport module.

Preferably, the fluid inlet is equipped with connection means of the Luer type, laid out in order to allow connection of a container such as a syringe. Advantageously, the fluid outlet is equipped with connection means, such as of the Luer type, laid out in order to allow the connection of an extension, one of the ends of which is equipped with means for connecting to a component forming a catheter.

Preferentially, the preparation and/or administration unit includes at least one scale, and the first control means are designed in order to control the displacement of the obturation means associated with each administration reservoir, and optionally with each dilution reservoir, between their first and second positions depending on the value measured by said scales.

According to an embodiment, the administration line includes a monitoring chamber positioned downstream from the mixing chamber, the monitoring chamber being equipped with monitoring means laid out for monitoring the quality of the fluid from the mixing chamber.

Advantageously, the transport module may include thermal insulation means in order to thermally protect the fluids contained in the different reservoirs housed in the transport module.

Anyhow, the invention will be well understood with the description which follows, referring to the appended schematic drawing illustrating as a non-limiting example, an embodiment of this preparation and/or administration system.

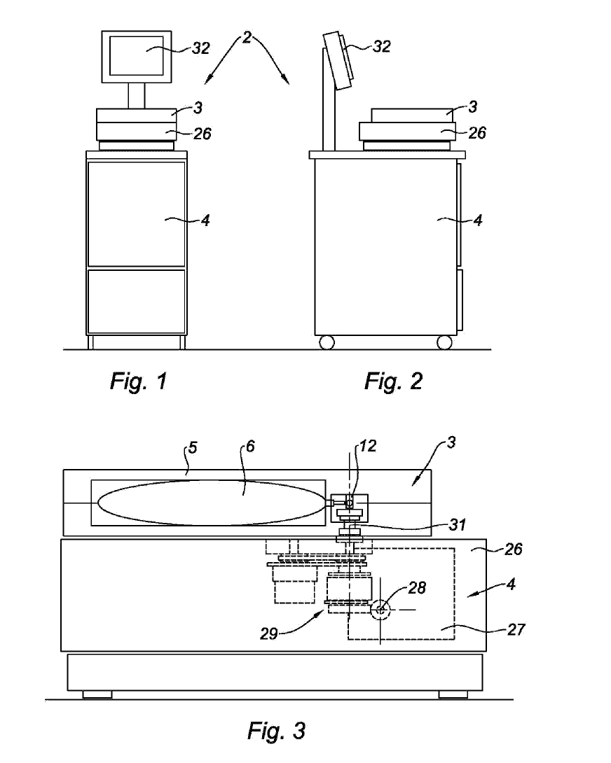

FIGS. 1 and 2 are schematic front and side views of a system for preparing and/or administering fluids according to the invention.

FIG. 3 is a partial view at an enlarged scale, of the preparation and/or administration system of FIG. 1.

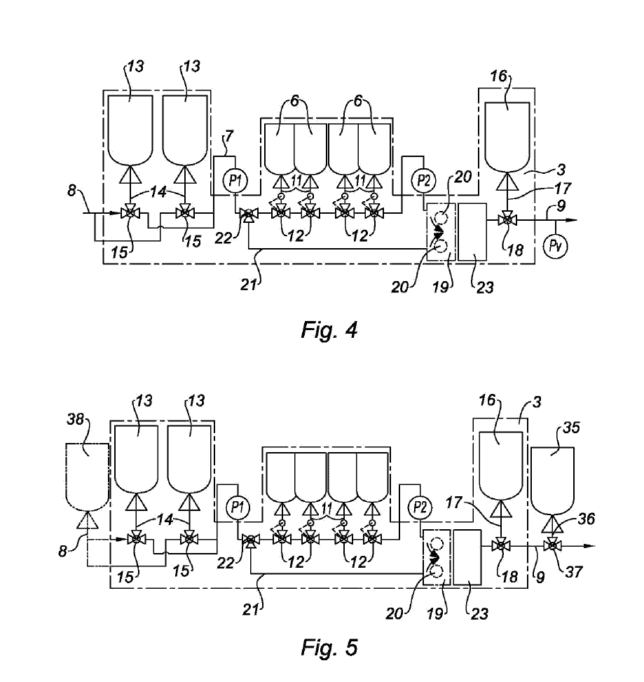

FIGS. 4 and 5 are schematic views of the transport module of the system of FIG. 1 in two different operating positions.

FIGS. 1 and 2 illustrate a system for preparing and administering fluids 2 according to the invention comprising a transport module 3 and a unit for preparing and administering fluids 4 on which the intention is to position the transport module 3.

As shown more particularly in FIG. 3, the transport module 3 is made as a transport box equipped with a lid 5 which is moveable between positions for opening and closing the transport box. The transport box may for example be made in plastic.

As shown in FIGS. 4 and 5, the transport module 3 contains a plurality of administration reservoirs 6 each intended to contain a medical treatment fluid to be administered to a patient, an administration line 7 equipped with a fluid inlet and outlet 8, 9 accessible from the outside of the transport module 3, and a plurality of connecting lines 11 each connecting one of the administration reservoirs 6 to the administration line 7.

Each connecting line 11 includes a 3-way valve 12 which is moveable between a first position in which the corresponding administration reservoir 6 is fluidically isolated from the administration line 7 and a second position in which the corresponding administration reservoir 6 is fluidically connected to the administration line 7.

Preferably, the fluid inlet 8 is equipped with connection means of the Luer type laid out in order to for example allow connection of a container, such as a syringe, and the fluid outlet 9 is equipped with connection means of the Luer type for example laid out in order to allow connection of an extension equipped with means for connecting to a component forming a catheter.

The transport module 3 also contains two dilution reservoirs 13 intended to respectively contain normal saline and glucose and two dilution lines 14 each connecting one of the dilution reservoirs 13 to the administration line 7 upstream from the administration reservoirs 6. Each dilution line 14 includes a 3-way valve 15 which is moveable into a first position in which the corresponding dilution reservoir 13 is fluidically isolated from the administration line 7 and a second position in which the corresponding dilution reservoir 13 is fluidically connected to the administration line 7.

The transport module 3 further contains a recovery reservoir 16 and a recovery line 17 connecting the recovery reservoir 16 to the administration line 7 upstream from the fluid outlet 9. The recovery line 17 includes a 3-way valve 18 which is moveable between a first position in which the recovery reservoir 16 is fluidically isolated from the administration line 7 and a second position in which the recovery reservoir 16 is fluidically connected to the administration line 7.

The administration line 7 comprises a mixing chamber 19 positioned downstream from the administration reservoir 6 and upstream from the recovery reservoir 16, the mixing chamber 19 advantageously including means 20 for mixing the different fluids contained in the latter.

The transport module 3 further includes a bypass line 21, one of the ends of which is connected to the mixing chamber 19 and the other end of which is connected to the administration line 7 upstream from the administration reservoir 6 and downstream from the dilution reservoir 13. The bypass line 21 includes a 3-way valve 22 which is moveable between a first position in which fluid flow through the bypass line 21 is prevented and a second position in which fluid flow through the bypass line 21 is allowed.

The administration line 7 further includes a volumetric pump P1 positioned between the dilution reservoirs 13 and the administration reservoirs 6, and a volumetric pump P2 positioned between the administration reservoirs 6 and the mixing chamber 19. These volumetric pumps P1, P2 allow the flow rate to be controlled in the administration line 7 of the different fluids contained in the administration and dilution reservoirs.

The administration line 7 also advantageously includes a monitoring chamber 23 positioned between the mixing chamber 19 and the recovery reservoir 16, the monitoring chamber 23 being equipped with monitoring means (not shown in the figures) laid out for monitoring the quality of the fluid from the mixing chamber 19.

Preferably, the different reservoirs 6, 13, 16 contained in the transport module 3 are flexible, and advantageously formed by perfusion bags.

The transport module 3 also includes locking means (not shown in the figures) of the lid 5 which are moveable between a locking position of the lid in its closing position and a position for unlocking the lid.

According to an embodiment of the invention, the transport module 3 may comprise instead of the volumetric pumps P1, P2 or as an addition to the latter, pressurization means (not shown in the figures) laid out so as to maintain a predetermined pressure on the outer walls of the administration reservoirs 6 housed in the transport module.

The preparation and administration unit 4 is made as a moveable trolley and includes first control means laid out for independently controlling the displacements of the 3-way valves of the recovery, bypass, connecting and dilution lines between their first and second positions. The first control means are capable of controlling the displacements of the different 3-way valves only in an operating position of the preparation and administration system 2 in which the transport module 3 is positioned on the preparation and administration unit 4.

The first control means include a control box 26 containing monitoring means (not shown in the figures), and actuation means driven by the monitoring means and laid out in order to mechanically cooperate with the 3-way valves of each connecting line when the transport module 3 is positioned on the preparation and administration unit 4 on the one hand, and for displacing the latter between their first and second positions on the other hand. The monitoring means advantageously include a micorprocessor. The actuation means preferably include an electric motor 27, the output shaft of which is designed in order to drive into rotation a wormscrew 28 coupled with a clutch system 29, the clutch system 29 being designed so as to drive into rotation a plurality of actuation members 31 each intended to cooperate with one of the 3-way valves of the transport module 3 so as to cause the latter to pivot between these first and second positions.

The microprocessor and the electric motor 27 are advantageously supplied with electric power through the mains and/or through a backup battery for example housed in the control box 26 and intended to make the preparation and administration unit 4 self-contained and insensitive to any power cut.

The preparation and administration unit 4 also includes a barcode reader and a barcode printer intended to print a label to be positioned on the transport module 3, the barcode of the latter containing data notably relating to the patient and to the contents of each reservoir of the transport module 3.

The preparation and administration unit 4 includes display and input means, such as a touch screen 32, and a central data processing unit housed in the trolley and including means for storing data intended for storing the inputted data with the touch screen 32 and the barcode reader.

The preparation and administration unit 4 also includes second control means (not shown in the figures) laid out so as to control the displacements of the means for locking the lid 5 of the transport module 3 between their locking and unlocking positions when the latter is positioned on the preparation and administration unit 4.

The preparation and administration unit 4 further includes means for locking the transport module 3 on the latter.

The preparation and administration unit 4 may advantageously include at least one electronic pair of scales. The further control means are then designed in order to control the displacement of the 3-way valves of each dilution and administration line between their first and second positions, depending on the value measured by said scales.

According to an embodiment of the invention, the transport module 3 is equipped with an identification module, preferably an RFID identification chip, and the preparation and administration unit 4 includes contactless interactive means intended to detect and identify the identification module borne by a transport module 3 when the latter is positioned on the preparation and administration unit 4, and data transfer means intended to transmit characteristic data of a detected and identified identification module to the central data processing unit. The data transferred by the transfer means are stored in the data storage means of the central unit.

According to an embodiment of the invention, the preparation and administration unit 4 comprises audio and/or visual alarm means indicating to an operator any malfunction of the unit and/or the transport module.

According to an embodiment of the invention, the central unit comprises means for time-stamping the data transferred to the central unit. Therefore it is possible to specifically record the date and time of the beginning and of the end of each preparation and administration cycle and the date and the time of each positioning and withdrawal of a transport module on said preparation and administration unit 4.

A method for preparing fluids according to the invention will now be described. It should be noted that in the initial position, the different 3-way valves contained in a transport module 3 are preferably in their first position.

The method for preparing fluids according to the invention comprises the following steps:

a) providing a preparation and administration unit 4,

b) identifying the patient with inputting means 3 or with the barcode reader of said unit 4,

c) positioning a transport module 3 on the preparation and administration unit 4,

d) locking the transport module 3 on the preparation and administration unit 4,

e) locking the lid 5 of the transport module 3,

f) connecting a vacuum pump PV to the fluid outlet 9 of the transport module 3 and applying a vacuum to the administration line 7,

g) identifying a container, such as a syringe, equipped with a barcode and containing a predetermined amount of a first medical treatment liquid, by means of the barcode reader, and connecting said container to the fluid inlet 8 of the transport module 3,

h) controlling the displacement of the 3-way valve 12 of one of the connecting lines 11 in its second position, filling the corresponding administration reservoir 6 with the first medical treatment liquid, controlling the displacement of said 3-way valve 12 into its first position, and disconnecting the container from the fluid inlet 8 of the transport module,

i) identifying a container, such as a syringe, equipped with a barcode and containing a predetermined amount of rinsing fluid, by means of the barcode reader, connecting said container to the fluid inlet 8 of the transport module 3, controlling the displacement of the 3-way valve 18 of the recovery line 17 in its second position, rinsing the administration line 7 with the rinsing fluid, controlling the displacement of the 3-way valve 18 of the recovery line in its first position, and disconnecting the container from the fluid inlet 8 of the transport module 3.

j) repeating steps g) to i) so as to fill the different administration reservoirs 6 with different medical treatment liquids,

k) connecting a container, such as syringe, containing a predetermined amount of normal saline to the fluid inlet 8 of the transport module,

l) controlling the displacement of the 3-way valve 15 of one of the dilution lines 14 in its second position, filling the corresponding dilution reservoir 13 with normal saline, controlling the displacement of said 3-way valve 15 in its first position, and disconnecting the container from the fluid inlet 8 of the transport module 3,

m) connecting a container, such as a syringe, containing a predetermined amount of glucose to the fluid inlet 8 of the transport module,

n) controlling the displacement of the 3-way valve 15 of the other dilution line 14 in its second position, filling the corresponding dilution reservoir 13 with glucose, controlling the displacement of said 3-way valve in its first position, and disconnecting the container from the fluid inlet 8 of the transport module 3,

o) printing a label with the barcode printer and positioning said label on the treatment module 3,

p) identifying the label positioned on the transport module 3 with the barcode reader,

q) unlocking the transport module 3 of the preparation and administration unit 4.

According to an embodiment of the preparation method, the latter comprises a step prior to step g) which consists of connecting a volumetric pump to the fluid inlet 8 so as to facilitate the filling of the different reservoirs housed in the transport module, and more particularly the dilution reservoirs 13.

According to an embodiment of the preparation method, the latter comprises a step prior to each step for identifying a container, which consists of weighing said container with electronic scales, the displacement of the 3-way valve of the corresponding dilution or administration line in its second position being controlled exclusively if the weight of the container is equal to a predetermined value.

According to an embodiment of the preparation method, the step for disconnecting a container from the fluid inlet 8 of the transport module 3 may be carried out before the step for controlling the displacement of the 3-way valve of the corresponding line into its first position.

A method for administering fluids to a patient will now be described.

The method for administering fluids according to the invention comprises the following steps:

a) providing a preparation and administration unit 4,

b) positioning a transport module 3, the reservoirs of which have been filled beforehand on the preparation and administration unit 4,

c) identifying the label positioned on the transport module 3, by means of the barcode reader of said unit,

d) identifying the patient with the help of input means or a barcode reader of said unit,

e) locking the transport module 3 on the preparation and administration unit 4,

f) connecting an extension to the fluid outlet 9 of the transport module 3,

g) purging the administration line 7 and the extension by means of a dilution fluid contained in one of the dilution reservoirs 13 by controlling the displacement of the corresponding 3-way valve 15 into its second position, and then controlling the displacement of said 3-way valve into its first position,

h) connecting the extension to a component forming a catheter connected to a patient,

i) controlling the displacement of the 3-way valve 12 of one of the connecting lines 11 into its second position, administering to the patient the medical treatment fluid contained in the corresponding administration reservoir 6, and controlling the displacement of said 3-way valve into its first position,

j) controlling the displacement of the 3-way valve of one of the dilution lines 14 into its second position, rinsing the administration line 7 and the extension with the fluid contained in the corresponding dilution reservoir 13, and controlling the displacement of said 3-way valve 15 into its first position,

k) repeating steps i) and j) so as to administer to the patient the different medical treatment fluids contained in the different administration reservoirs 6,

l) recording the date and time of the end of the administration cycle by time-stamping means,

m) unlocking the lid 5 of the transport module 3 and unlocking the transport module 3 of the preparation and administration unit 4,

n) disconnecting the extension from the fluid outlet 9.

According to an embodiment of the preparation method, the latter comprises a step prior to each step for administering a medical treatment fluid to the patient which consists of diluting said medical treatment fluid in the mixing chamber 19 with one of the dilution fluids contained in the dilution reservoirs 13, and then mixing both of these fluids in the mixing chamber 19 with mixing means 20.

According to an embodiment of the preparation method, the latter comprises a step consisting of monitoring in the monitoring chamber 23 the quality of the fluid from the mixing chamber 19 before its administration to the patient, and of allowing this administration only if the quality of the fluid is satisfactory.

According to another embodiment of the preparation, the latter comprises a step consisting of connecting a backup reservoir 35 containing a rinsing fluid to the fluid outlet 9 of the transport module 3 via a backup line 36 equipped with a 3-way valve 37 which may be displaced manually between a first position in which the backup reservoir 35 is fluidically isolated from the fluid outlet 9 and a second position in which the backup reservoir 35 is fluidically connected to the fluid outlet 9. The presence of such a backup reservoir 35 gives the possibility of ensuring rinsing of the extension in the case of failure of the transport module 3 and/or of the preparation and administration unit 4.

According to still another embodiment of the preparation method, the latter comprises a step consisting of connecting a reservoir 38 containing a dilution fluid to the fluid inlet 8 of the transport module 3. The presence of such a reservoir gives the possibility of compensating for a possible lack of dilution fluid inside the transport module 3.

As this is clear, the invention is not limited to the single embodiment of this system for preparing and/or administering fluids, described above as an example, it encompasses on the contrary all the alternative embodiments.

1. A system for preparing and/or administering medical treatment fluids comprising:
at least one transport module in which are housed at least one administration reservoir intended to contain a medical treatment fluid to be administered to a patient, an administration line equipped with a fluid inlet and outlet accessible from the outside of the transport module and to which is connected each administration reservoir, and obturation means associated with each administration reservoir, the obturation means associated with each administration reservoir being moveable between a first position in which the corresponding administration reservoir is fluidically isolated from the administration line and a second position in which the corresponding administration reservoir is fluidically connected to the administration line, and
at least one preparation and/or administration unit on which the intention is to position the transport module, the preparation and/or administration unit including first control means laid out for controlling the displacements of the obturation means associated with each administration reservoir between their first and second positions.

2. The system according to claim 1, wherein the first control means are laid out so as to control the displacements of the obturation means associated with each administration reservoir only in an operating position of the preparation and/or administration system in which the transport module is positioned on the preparation and/or administration unit.

3. The system according to claim 1, wherein the first control means include monitoring means and actuation means driven by the monitoring means and laid out so as to mechanically cooperate with the obturation means associated with each administration reservoir when the transport module is positioned on the preparation and/or administration unit and for displacing the latter between their first and second positions.

4. The system according to claim 1, wherein the obturation means associated with each administration reservoir include a 3-way valve.

5. The system according to claim 1, wherein the first control means are laid out so as to be electrically coupled with the obturation means associated with each administration reservoir when the transport module is positioned on the preparation and/or administration unit.

6. The system according to claim 1, wherein the transport module includes a lid moveable between opening and closing positions of the transport module, and means for locking the lid which are moveable between a position for locking the lid in its closing position and a position for unlocking the lid.

7. The system according to claim 6, wherein the preparation and/or administration unit comprises second control means laid out for controlling the displacements of the locking means between their locking and unlocking positions.

8. The system according to claim 1, wherein it includes means for locking the transport module on the preparation and/or administration unit.

9. The system according to claim 1, wherein the transport module further contains at least one dilution reservoir connected to the administration line upstream from the administration reservoirs and intended to contain a dilution fluid, such as normal saline or glucose, and obturation means associated with each dilution reservoir, the obturation means associated with each dilution reservoir being moveable between a first position in which the corresponding dilution reservoir is fluidically isolated from the administration line and a second position in which the corresponding dilution reservoir is fluidically connected to the administration line and wherein the first control means are laid out so as to control the displacements of the obturation means associated with each dilution reservoir between their first and second positions.

10. The system according to claim 1, wherein the administration line comprises a mixing chamber positioned downstream from the administration reservoirs and upstream from the fluid outlet of the transport module.

11. The system according to claim 9, wherein the transport module includes a bypass line, one of the ends of which is connected to the mixing chamber and the other end of which is connected to the administration line upstream from the administration reservoirs and downstream from said at least one dilution reservoir, the bypass line including obturation means which are moveable between a first position in which the obturation means prevent fluid flow through the bypass line and a second position in which the obturation means allow fluid flow through the bypass line, and wherein the first control means are laid out so as to control the displacements of the obturation means of the bypass line between their first and second positions.

12. The system according to claim 11, wherein the administration line includes a volumetric pump positioned between the administration reservoirs and the mixing chamber.

13. The system according to claim 9, wherein the administration line includes a volumetric pump positioned between said at least one dilution reservoir and the administration reservoirs.

14. The system according to claim 1, wherein the transport module comprises a recovery reservoir connected to the administration line upstream from the fluid outlet and preferably downstream from the mixing chamber, and obturation means which are moveable between a first position in which the recovery reservoir is fluidically isolated from the administration line and a second position in which the recovery reservoir is fluidically connected to the administration line, and wherein the first control means are laid out for controlling the displacements of the obturation means associated with the recovery reservoir between their first and second positions.

15. The system according to claim 1, wherein the transport module is equipped with an identification module and wherein the preparation and/or administration unit includes contactless interactive means intended to detect and identify the identification module when the transport module is positioned on the preparation and/or administration unit.
